SEQUENCE LISTING (1 sequence)

SEQ ID NO: 1 - protein, 87 aa, unidentified
FVNQHLCGSHLVEALYLVCGERGFFYTPKTRREAEDLQGSLQPLALEGSLQGSLQPLALEGSLQKRGIVEQCCTSICSLYQLENYCN

(19) United States
(12) Patent Application Publication
(10) Pub. No.: US 2025/0123288 A1
(43) Pub. Date: Apr. 17, 2025

(54) METHODS FOR THE SIMULTANEOUS DETECTION AND QUANTIFICATION OF INSULIN, PROINSULIN, PROINSULIN METABOLIC INTERMEDIATES, C-PEPTIDE, AND C-PEPTIDE VARIANTS BY MASS SPECTROMETRY

(71) Applicant: Quest Diagnostics Investments LLC, Secaucus, NJ (US)

(72) Inventors: Timothy S. Collier, Moreland Hills, OH (US); Ryan W. Pearce, North Ridgeville, OH (US); Michael J. McPhaul, Capistrano Beach, CA (US)

(21) Appl. No.: 18/910,466

(22) Filed: Oct. 9, 2024

Related U.S. Application Data
(60) Provisional application No. 63/589,367, filed on Oct. 11, 2023.

(51) Int. Cl. G01N 33/68; G01N 33/53; G01N 33/74

(52) U.S. Cl. CPC G01N 33/6848; G01N 33/53; G01N 33/74; G01N 2333/62

(57) ABSTRACT

Methods are described for determining the amount of an analyte in a sample, wherein the analyte comprises insulin, proinsulin, a metabolic intermediate of proinsulin, C-peptide, a mutant C-peptide, or a mixture of any two or more thereof. Provided herein are mass spectrometric methods for detecting and quantifying an analyte (e.g., insulin, proinsulin, a metabolic intermediate of proinsulin, C-peptide, a mutant C-peptide, or a mixture of any two or more thereof) in a biological sample utilizing enrichment and/or purification methods coupled with mass spectrometric techniques.

### CROSS-REFERENCE TO RELATED APPLICATIONS

This application claims priority to U.S. Provisional Patent Application No. 63/589,367, filed Oct. 11, 2023, the entire disclosure of which is incorporated herein by reference in its entirety, for any and all purposes.

The instant application contains a Sequence Listing which has been submitted electronically in XML format and is hereby incorporated by reference in its entirety. Said XML copy, created on Nov. 7, 2024, is named 034827-5403_SL.txt and is 2,111 bytes in size.

### BACKGROUND

Insulin is a hormone that is central to regulating carbohydrate and fat metabolism in the body. Aberrant levels of insulin indicate glycemic disorders and/or insulin resistant syndromes such as diabetes. Diabetes and its complications represent a major public health issue.

Proinsulin is the prohormone precursor to insulin. The most common method for measuring proinsulin is immunoassay, but this method suffers from cross-reactivity with proinsulin intermediates such as des-31,32-proinsulin and des-64,65-proinsulin.

Thus, there remains a need for new measurements with improved reliability and accuracy.

### SUMMARY

In one aspect, provided herein are methods for determining an amount of an analyte in a sample by mass spectrometry, the method comprising:
immunocapturing the analyte from the sample;
subjecting the immunocaptured analyte to an ionization source under conditions suitable to generate one or more ions detectable by mass spectrometry;
determining an amount of the one or more ions by mass spectrometry; and
determining the amount of the analyte in the sample from the amount of the one or more ions;
wherein
   the analyte comprises insulin, proinsulin, a metabolic intermediate of proinsulin, C-peptide, a mutant C-peptide, or a mixture of any two or more thereof;
   the metabolic intermediate of proinsulin is des-31,32-proinsulin, des-64,65-proinsulin, or a mixture thereof; and
   the mutant C-peptide is C-peptide with one additional arginine residue on C-terminal end (C-peptide+R), C-peptide with two additional arginine residues on C-terminal end (C-peptide+RR), or a mixture thereof.

In another aspect, provided herein are methods for determining an amount of proinsulin in a sample, the method comprising:
immunocapturing proinsulin from the sample;
subjecting the immunocaptured proinsulin to an ionization source under conditions suitable to generate one or more proinsulin ions detectable by mass spectrometry;
determining an amount of the one or more proinsulin ions by mass spectrometry; and
determining the amount of proinsulin in the sample from the amount of the one or more proinsulin ions.

In another aspect, provided herein are methods for determining an amount of a metabolic intermediate of proinsulin in a sample, the method comprising:
immunocapturing the metabolic intermediate from the sample;
subjecting the immunocaptured metabolic intermediate to an ionization source under conditions suitable to generate one or more metabolic intermediate ions detectable by mass spectrometry;
determining an amount of the one or more metabolic intermediate ions by mass spectrometry; and
determining the amount of the metabolic intermediate of proinsulin in the sample from the amount of the one or more metabolic intermediate ions;
wherein
   the metabolic intermediate of proinsulin is des-31,32-proinsulin, des-64,65-proinsulin, or a mixture thereof.

In another aspect, provided herein are methods determining an amount of a mutant C-peptide in a sample, the method comprising:
immunocapturing the mutant C-peptide from the sample;
subjecting the immunocaptured mutant C-peptide to an ionization source under conditions suitable to generate one or more mutant C-peptide ions detectable by mass spectrometry;
determining an amount of the one or more mutant C-peptide ions by mass spectrometry; and
determining the amount of the mutant C-peptide in the sample from the amount of the one or more mutant C-peptide ions;
wherein
   the mutant C-peptide is C-peptide with one additional arginine residue on C-terminal end (C-peptide+R), C-peptide with two additional arginine residues on C-terminal end (C-peptide+RR), or a mixture thereof.

In some embodiments, the analyte comprises proinsulin. In some embodiments, the one or more ions comprise one or more proinsulin ions. In some embodiments, the one or more proinsulin ions comprise a proinsulin precursor ion with a mass-to-charge ratio (m/z) of 1342.20±0.5. In some embodiments, the one or more proinsulin ions comprise one or more proinsulin fragment ions selected from a group consisting of ions with m/z of 183.30±0.5, 120.20±0.5, and 219.30=0.5.

In some embodiments, the analyte comprises a metabolic intermediate of proinsulin. In some embodiments, the metabolic intermediate of proinsulin comprises des-31,32-proinsulin. In some embodiments, the one or more ions comprise one or more des-31,32-proinsulin ions. In some embodiments, the one or more des-31,32-proinsulin ions comprise a des-31,32-proinsulin precursor ion with m/z of 1299.50±0.5. In some embodiments, the one or more des-31,32-proinsulin ions comprise one or more des-31,32-proinsulin fragment ions selected from a group consisting of ions with m/z of 129.20±0.5, 226.20±0.5, and 183.25±0.5.

In some embodiments, the metabolic intermediate of proinsulin comprises des-64,65-proinsulin. In some embodiments, the one or more ions comprise one or more des-64,65-proinsulin ions. In some embodiments, the one or more des-64,65-proinsulin ions comprise a des-64,65-proinsulin precursor ion with m/z of 1304.30±0.5. In some embodiments, the one or more des-64,65-proinsulin ions comprise one or more des-64,65-proinsulin fragment ions selected from a group consisting of ions with m/z of 147.15±0.5, 130.15±0.5, and 183.20=0.5.

In some embodiments, the analyte comprises a mutant C-peptide. In some embodiments, the mutant C-peptide comprises C-peptide with one additional arginine residue on C-terminal end (C-peptide+R). In some embodiments, the one or more ions comprise one or more C-peptide+R ions. In some embodiments, the one or more C-peptide+R ions comprise a precursor C-peptide+R ion with m/z of 1059.20±0.5. In some embodiments, the one or more C-peptide+R ions comprise one or more C-peptide+R fragment ions selected from a group consisting of ions with m/z of 966.40±0.5, 260.00±0.5, and 844.30±0.5. In some embodiments, the mutant C-peptide comprises C-peptide with two additional arginine residues on C-terminal end (C-peptide+RR). In some embodiments, the one or more ions comprise one or more C-peptide+RR ions. In some embodiments, the one or more C-peptide+RR ions comprise a C-peptide+RR precursor ion with m/z of 1111.50±0.5. In some embodiments, the one or more C-peptide+RR ions comprise one or more C-peptide+RR fragment ions selected from a group consisting of ions with m/z of 896.50±0.5, 499.90±0.5, and 260.30±0.5.

In some embodiments, the analyte comprises insulin. In some embodiments, the one or more ions comprise one or more insulin ions. In some embodiments, the one or more insulin ions comprise an insulin precursor ion with m/z of 1162.30±0.5. In some embodiments, the one or more insulin ions comprise one or more insulin fragment ions selected from a group consisting of ions with m/z of 226.20±0.5, 136.10±0.5, and 345.20±0.5.

In some embodiments, the analyte comprises C-peptide. In some embodiments, the one or more ions comprise one or more C-peptide ions. In some embodiments, the one or more C-peptide ions comprise a C-peptide precursor ion with m/z of 1007.70±0.5. In some embodiments, the one or more C-peptide ions comprise one or more C-peptide fragment ions selected from a group consisting of ions with m/z of 927.50±0.5, 646.40±0.5, and 533.30±0.5.

In some embodiments, the analyte is not insulin, C-peptide, or a mixture of insulin and C-peptide.

In some embodiments, the sample comprises a plasma or serum sample.

In some embodiments, the ionization source is an electrospray (ESI) ionization source. In some embodiments, ionization is in positive ion mode.

In some embodiments, the sample is subjected to acidic conditions prior to mass spectrometry. In some embodiments, subjecting said sample to acidic conditions comprises subjecting said sample to formic acid. In some embodiments, said sample is subjected to basic conditions prior to mass spectrometry. In some embodiments, subjecting said sample to basic conditions comprises subjecting said sample to trizma and/or ethanol.

In some embodiments, the sample is delipidated prior to quantitation by mass spectrometry.

In some embodiments, a method described herein further comprises purifying the sample prior to mass spectrometry. In some embodiments, said purifying comprises subjecting the sample to liquid chromatography. In some embodiments, liquid chromatography comprises high performance liquid chromatography (HPLC) or high turbulence liquid chromatograph (HTLC). In some embodiments, said purifying comprises subjecting a sample to solid phase extraction (SPE).

In some embodiments, the mass spectrometry is tandem mass spectrometry, high resolution mass spectrometry, or high resolution/high accuracy mass spectrometry.

In some embodiments, the immunocapture comprises using antibodies (e.g., monoclonal antibodies). In some embodiments, the antibodies are immobilized on magnetic beads. In some embodiments, a method described herein further comprises washing and eluting the materials immunocaptured on magnetic beads.

### BRIEF DESCRIPTION OF THE DRAWINGS

FIG. 1 shows example chromatogram of the detectable analytes in a multiplexed LC-MS assay as described in Example 1.

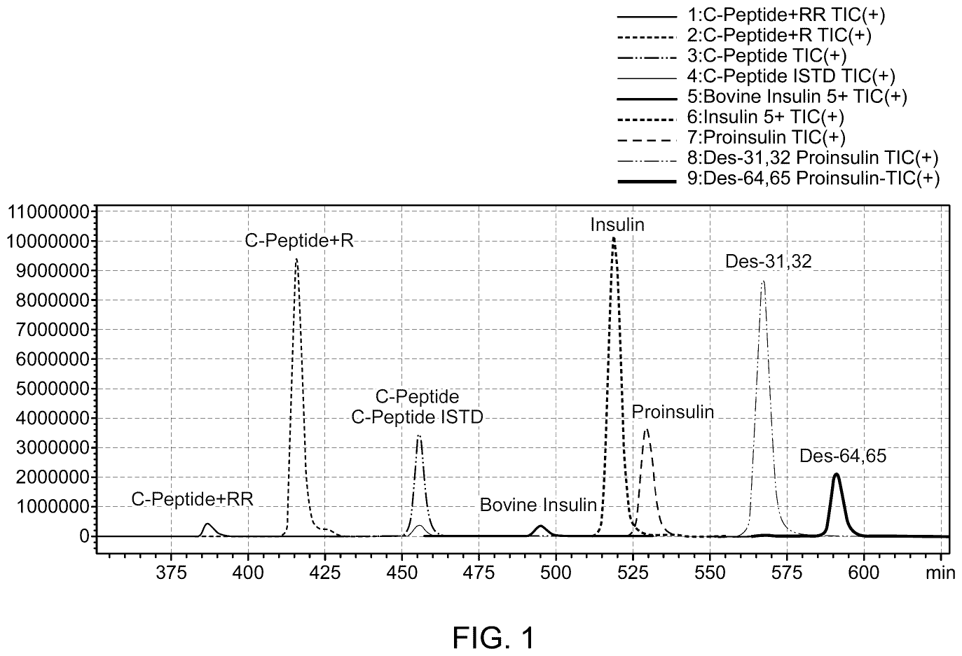

FIG. 2 shows a comparison between the proinsulin immunoassay method and the LC-MS method. A strong correlation between methods is observed (Spearman r=0.75).

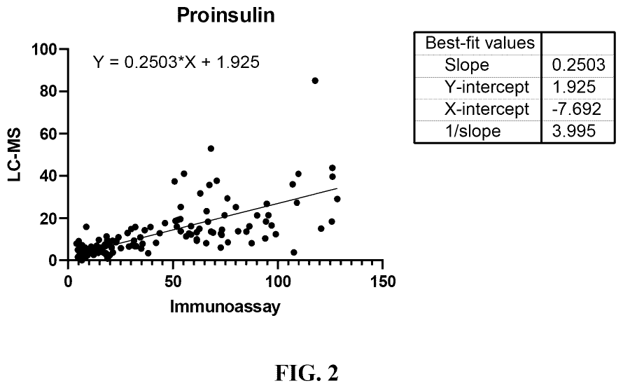

FIG. 3 shows the major and minor processing pathways of proinsulin, which produce 65, 66 split proinsulin, des-64,65 proinsulin, 32, 33 split proinsulin, des-31,32-proinsulin, C-peptide, and/or insulin.

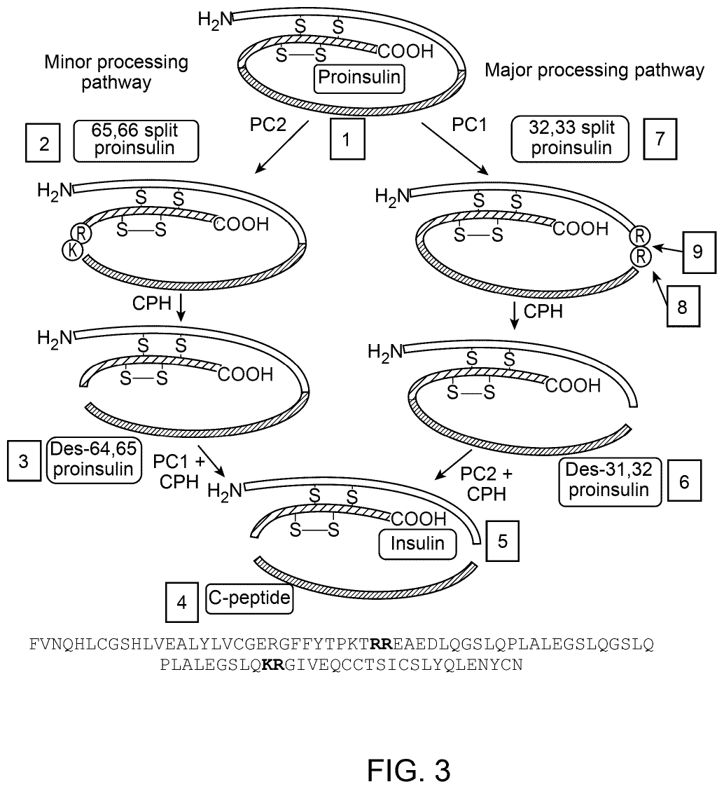

### DETAILED DESCRIPTION

### Definitions

In order for the present technology to be more readily understood, certain terms are first defined below. Additional definitions for the following terms and other terms are set forth throughout the specification. The publications and other reference materials referenced herein to describe the background of the technology and to provide additional detail regarding its practice are hereby incorporated by reference.

As used herein, unless otherwise stated, the singular forms “a,” “an,” and “the” include plural reference. Thus, for example, a reference to “a protein” includes a plurality of protein molecules.

As used herein, the terms “purification,” “purifying,” and “enriching” do not refer to removing all materials from the sample other than the analyte(s) of interest. Instead, these terms refer to a procedure that enriches the amount of one or more analytes of interest relative to other components in the sample that may interfere with detection of the analyte of interest. Purification of the sample by various means may allow relative reduction of one or more interfering substances, e.g., one or more substances that may or may not interfere with the detection of selected parent or daughter ions by mass spectrometry. Relative reduction as this term is used does not require that any substance, present with the analyte of interest in the material to be purified, is entirely removed by purification.

As used herein, the term “immunopurification” or “immunopurify” refers to a purification procedure that utilizes antibodies, including polyclonal or monoclonal antibodies, to enrich the one or more analytes of interest. Immunopurification can be performed using any of the immunopurification methods well known in the art. Often the immunopurification procedure utilizes antibodies bound, conjugated or otherwise attached to a solid support, for example a column, well, tube, gel, capsule, particle or the like. Immunopurification as used herein includes without limitation procedures often referred to in the art as immunoprecipitation, as well as procedures often referred to in the art as affinity chromatography or immunoaffinity chromatography.

As used herein, the term “immunoparticle” refers to a capsule, bead, gel particle or the like that has antibodies bound, conjugated or otherwise attached to its surface (either on and/or in the particle). In certain preferred embodiments, immunoparticles are sepharose or agarose beads. In alternative preferred embodiments, immunoparticles comprise glass, plastic or silica beads, or silica gel.

As used herein, the term “anti-insulin antibody” refers to any polyclonal or monoclonal antibody that has an affinity for insulin, and the term “anti-C-peptide antibody” refers to any polyclonal or monoclonal antibody that has an affinity for C-peptide. In various embodiments the specificity of the antibodies (e.g., insulin antibodies or anti-C-peptide antibodies) to chemical species other than the target antigen (e.g., insulin or C-peptide) may vary. For example, in some embodiments, the antibodies are specific for the target antigen and thus have little or no affinity for chemical species other than the target antigen; whereas in some other embodiments, the antibodies are non-specific and thus bind certain chemical species other than the target antigen.

As used herein, the term “sample” refers to any sample that may contain an analyte of interest. As used herein, the term “body fluid” means any fluid that can be isolated from the body of an individual. For example, “body fluid” may include blood, plasma, serum, bile, saliva, urine, tears, perspiration, and the like. In preferred embodiments, the sample comprises a body fluid sample from human; preferably plasma or serum.

As used herein, the term “solid phase extraction” or “SPE” refers to a process in which a chemical mixture is separated into components as a result of the affinity of components dissolved or suspended in a solution (i.e., mobile phase) for a solid through or around which the solution is passed (i.e., solid phase). In some instances, as the mobile phase passes through or around the solid phase, undesired components of the mobile phase may be retained by the solid phase resulting in a purification of the analyte in the mobile phase. In other instances, the analyte may be retained by the solid phase, allowing undesired components of the mobile phase to pass through or around the solid phase. In these instances, a second mobile phase is then used to elute the retained analyte off of the solid phase for further processing or analysis. SPE, including TFLC, may operate via a unitary or mixed mode mechanism. Mixed mode mechanisms utilize ion exchange and hydrophobic retention in the same column; for example, the solid phase of a mixed-mode SPE column may exhibit strong anion exchange and hydrophobic retention; or may exhibit strong cation exchange and hydrophobic retention.

Generally, the affinity of a SPE column packing material for an analyte may be due to any of a variety of mechanisms, such as one or more chemical interactions or an immunoaffinity interaction. In some embodiments, SPE of insulin is conducted without the use of an immunoaffinity column packing material. That is, in some embodiments, insulin is purified from a sample by a SPE column that is not an immunoaffinity column.

As used herein, the term “chromatography” refers to a process in which a chemical mixture carried by a liquid or gas is separated into components as a result of differential distribution of the chemical entities as they flow around or over a stationary liquid or solid phase.

As used herein, the term “liquid chromatography” or “LC” means a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). Examples of “liquid chromatography” include reverse phase liquid chromatography (RPLC), high performance liquid chromatography (HPLC), and turbulent flow liquid chromatography (TFLC) (sometimes known as high turbulence liquid chromatography (HTLC) or high through-put liquid chromatography).

As used herein, the term “high performance liquid chromatography” or “HPLC” (sometimes known as “high pressure liquid chromatography”) refers to liquid chromatography in which the degree of separation is increased by forcing the mobile phase under pressure through a stationary phase, typically a densely packed column.

As used herein, the term “turbulent flow liquid chromatography” or “TFLC” (sometimes known as high turbulence liquid chromatography or high throughput liquid chromatography) refers to a form of chromatography that utilizes turbulent flow of the material being assayed through the column packing as the basis for performing the separation. TFLC has been applied in the preparation of samples containing two unnamed drugs prior to analysis by mass spectrometry. See, e.g., Zimmer et al., J Chromatogr A 854:23-35 (1999); see also, U.S. Pat. Nos. 5,968,367, 5,919,368, 5,795,469, and 5,772,874, which further explain TFLC. Persons of ordinary skill in the art understand “turbulent flow”. When fluid flows slowly and smoothly, the flow is called “laminar flow”. For example, fluid moving through an HPLC column at low flow rates is laminar. In laminar flow the motion of the particles of fluid is orderly with particles moving generally in substantially straight lines. At faster velocities, the inertia of the water overcomes fluid frictional forces and turbulent flow results. Fluid not in contact with the irregular boundary “outruns” that which is slowed by friction or deflected by an uneven surface. When a fluid is flowing turbulently, it flows in eddies and whirls (or vortices), with more “drag” than when the flow is laminar. Many references are available for assisting in determining when fluid flow is laminar or turbulent (e.g., Turbulent Flow Analysis: Measurement and Prediction, P. S. Bernard & J. M. Wallace, John Wiley & Sons, Inc., (2000); An Introduction to Turbulent Flow, Jean Mathieu & Julian Scott, Cambridge University Press (2001)).

As used herein, the term “gas chromatography” or “GC” refers to chromatography in which the sample mixture is vaporized and injected into a stream of carrier gas (as nitrogen or helium) moving through a column containing a stationary phase composed of a liquid or a particulate solid and is separated into its component compounds according to the affinity of the compounds for the stationary phase.

As used herein, the term “large particle column” or “extraction column” refers to a chromatography column containing an average particle diameter greater than about 50 μm. As used in this context, the term “about” means±10%.

As used herein, the term “analytical column” refers to a chromatography column having sufficient chromatographic plates to effect a separation of materials in a sample that elute from the column sufficient to allow a determination of the presence or amount of an analyte. Such columns are often distinguished from “extraction columns”, which have the general purpose of separating or extracting retained material from non-retained materials in order to obtain a purified sample for further analysis. As used in this context, the term “about” means±10%. In a preferred embodiment the analytical column contains particles of about 5 μm in diameter.

As used herein, the terms “on-line” and “inline”, for example as used in “on-line automated fashion” or “on-line extraction”, refers to a procedure performed without the need for operator intervention. In contrast, the term “off-line” as used herein refers to a procedure requiring manual intervention of an operator. Thus, if samples are subjected to precipitation and the supernatants are then manually loaded into an autosampler, the precipitation and loading steps are off-line from the subsequent steps. In various embodiments of the methods, one or more steps may be performed in an on-line automated fashion.

As used herein, the term “sample injection” refers to introducing an aliquot of a single sample into an analytical instrument, for example a mass spectrometer. This introduction may occur directly or indirectly. An indirect sample injection may be accomplished, for example, by injecting an aliquot of a sample into a HPLC column that is connected to a mass spectrometer in an on-line fashion.

As used herein, the term “same sample injection” with respect to multiple analyte analysis by mass spectrometry means that the ions for two or more different analytes are determined essentially simultaneously by measuring ions for the different analytes from the same (i.e. identical) sample injection.

As used herein, the term “mass spectrometry” or “MS” refers to an analytical technique to identify compounds by their mass. MS refers to methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ionizer, a mass analyzer, and an ion detector. In general, one or more molecules of interest are ionized, and the ions are subsequently introduced into a mass spectrometric instrument where, due to a combination of magnetic and electric fields, the ions follow a path in space that is dependent upon mass (“m”) and charge (“z”). See, e.g., U.S. Pat. No. 6,204,500, entitled “Mass Spectrometry From Surfaces;” U.S. Pat. No. 6,107,623, entitled “Methods and Apparatus for Tandem Mass Spectrometry;” U.S. Pat. No. 6,268,144, entitled “DNA Diagnostics Based On Mass Spectrometry;” U.S. Pat. No. 6,124,137, entitled “Surface-Enhanced Photolabile Attachment And Release For Desorption And Detection Of Analytes;” Wright et al., Prostate Cancer and Prostatic Diseases 1999, 2:264-76; and Merchant and Weinberger, Electrophoresis 2000, 21:1164-67.

As used herein, “high resolution/high accuracy mass spectrometry” refers to mass spectrometry conducted with a mass analyzer capable of measuring the mass to charge ratio of a charged species with sufficient precision and accuracy to confirm a unique chemical ion. Confirmation of a unique chemical ion is possible for an ion when individual isotopic peaks from that ion are readily discernable. The particular resolving power and mass accuracy necessary to confirm a unique chemical ion varies with the mass and charge state of the ion.

As used herein, the term “resolving power” or “resolving power (FWHM)” (also known in the art as “m/Δm_{50%}”) refers to an observed mass to charge ratio divided by the width of the mass peak at 50% maximum height (Full Width HalfMaximum, “FWHM”).

As used herein a “unique chemical ion” with respect to mass spectrometry refers a single ion with a single atomic makeup. The single ion may be singly or multiply charged.

As used herein, the term “accuracy” (or “mass accuracy”) with respect to mass spectrometry refers to potential deviation of the instrument response from the true m/z of the ion investigated. Accuracy is typically expressed in parts per million (ppm).

High resolution/high accuracy mass spectrometry methods may be conducted on instruments capable of performing mass analysis with FWHM of greater than 10,000, 15,000, 20,000, 25,000, 50,000, 100,000, or even more. Likewise, methods of the present invention may be conducted on instruments capable of performing mass analysis with accuracy of less than 50 ppm, 20 ppm, 15 ppm, 10 ppm, 5 ppm, 3 ppm, or even less. Instruments capable of these performance characteristics may incorporate certain orbitrap mass analyzers, time-of-flight (“TOF”) mass analyzers, or Fourier-transform ion cyclotron resonance mass analyzers.

The term “orbitrap” describes an ion trap consisting of an outer barrel-like electrode and a coaxial inner electrode. Ions are injected tangentially into the electric field between the electrodes and trapped because electrostatic interactions between the ions and electrodes are balanced by centrifugal forces as the ions orbit the coaxial inner electrode. As an ion orbits the coaxial inner electrode, the orbital path of a trapped ion oscillates along the axis of the central electrode at a harmonic frequency relative to the mass to charge ratio of the ion. Detection of the orbital oscillation frequency allows the orbitrap to be used as a mass analyzer with high accuracy (as low as 1-2 ppm) and high resolving power (FWHM) (up to about 200,000). A mass analyzer based on an orbitrap is described in detail in U.S. Pat. No. 6,995,364, incorporated by reference herein in its entirety. Use of orbitrap analyzers has been reported for qualitative and quantitative analyses of various analytes. See, e.g., U.S. Patent Application Pub. No. 2008/0118932 (filed Nov. 9, 2007); Bredehoft, et al., Rapid Commun. Mass Spectrom., 2008, 22:477-485; Le Breton, et al., Rapid Commun. Mass Spectrom., 2008, 22:3130-36; Thevis, et al., Mass Spectrom. Reviews, 2008, 27:35-50; Thomas, et al., J. Mass Spectrom., 2008, 43:908-15; Schenk, et al., BMC Medical Genomics, 2008, 1:41; and Olsen, et al., Nature Methods, 2007, 4:709-12.

As used herein, the term “operating in negative ion mode” refers to those mass spectrometry methods where negative ions are generated and detected. The term “operating in positive ion mode” as used herein, refers to those mass spectrometry methods where positive ions are generated and detected. In some embodiments, mass spectrometry is conducted in positive ion mode.

As used herein, the term “ionization” or “ionizing” refers to the process of generating an analyte ion having a net electrical charge equal to one or more electron units. Negative ions are those having a net negative charge of one or more electron units, while positive ions are those having a net positive charge of one or more electron units.

As used herein, the term “electron ionization” or “EI” refers to methods in which an analyte of interest in a gaseous or vapor phase interacts with a flow of electrons. Impact of the electrons with the analyte produces analyte ions, which may then be subjected to a mass spectrometry technique.

As used herein, the term “chemical ionization” or “CI” refers to methods in which a reagent gas (e.g. ammonia) is subjected to electron impact, and analyte ions are formed by the interaction of reagent gas ions and analyte molecules.

As used herein, the term “fast atom bombardment” or “FAB” refers to methods in which a beam of high energy atoms (often Xe or Ar) impacts a non-volatile sample, desorbing and ionizing molecules contained in the sample. Test samples are dissolved in a viscous liquid matrix such as glycerol, thioglycerol, m-nitrobenzyl alcohol, 18-crown-6 crown ether, 2-nitrophenyloctyl ether, sulfolane, diethanolamine, and triethanolamine. The choice of an appropriate matrix for a compound or sample is an empirical process.

As used herein, the term “matrix-assisted laser desorption ionization” or “MALDI” refers to methods in which a non-volatile sample is exposed to laser irradiation, which desorbs and ionizes analytes in the sample by various ionization pathways, including photo-ionization, protonation, deprotonation, and cluster decay. For MALDI, the sample is mixed with an energy-absorbing matrix, which facilitates desorption of analyte molecules.

As used herein, the term “surface enhanced laser desorption ionization” or “SELDI” refers to another method in which a non-volatile sample is exposed to laser irradiation, which desorbs and ionizes analytes in the sample by various ionization pathways, including photo-ionization, protonation, deprotonation, and cluster decay. For SELDI, the sample is typically bound to a surface that preferentially retains one or more analytes of interest. As in MALDI, this process may also employ an energy-absorbing material to facilitate ionization.

As used herein, the term “electrospray ionization” or “ESI,” refers to methods in which a solution is passed along a short length of capillary tube, to the end of which is applied a high positive or negative electric potential. Solution reaching the end of the tube is vaporized (nebulized) into a jet or spray of very small droplets of solution in solvent vapor. This mist of droplets flows through an evaporation chamber. As the droplets get smaller the electrical surface charge density increases until such time that the natural repulsion between like charges causes ions as well as neutral molecules to be released.

As used herein, the term “atmospheric pressure chemical ionization” or “APCI,” refers to mass spectrometry methods that are similar to ESI; however, APCI produces ions by ion-molecule reactions that occur within a plasma at atmospheric pressure. The plasma is maintained by an electric discharge between the spray capillary and a counter electrode. Then ions are typically extracted into the mass analyzer by use of a set of differentially pumped skimmer stages. A counterflow of dry and preheated N2 gas may be used to improve removal of solvent. The gas-phase ionization in APCI can be more effective than ESI for analyzing less-polar species.

The term “atmospheric pressure photoionization” or “APPI” as used herein refers to the form of mass spectrometry where the mechanism for the ionization of molecule Mis photon absorption and electron ejection to form the molecular ion M+. Because the photon energy typically is just above the ionization potential, the molecular ion is less susceptible to dissociation. In many cases it may be possible to analyze samples without the need for chromatography, thus saving significant time and expense. In the presence of water vapor or protic solvents, the molecular ion can extract H to form MH+. This tends to occur if M has a high proton affinity. This does not affect quantitation accuracy because the sum of M+ and MH+ is constant. Drug compounds in protic solvents are usually observed as MH+, whereas nonpolar compounds such as naphthalene or testosterone usually form M+. See, e.g., Robb et al., Anal. Chem. 2000, 72 (15): 3653-3659.

As used herein, the term “inductively coupled plasma” or “ICP” refers to methods in which a sample interacts with a partially ionized gas at a sufficiently high temperature such that most elements are atomized and ionized.

As used herein, the term “field desorption” refers to methods in which a non-volatile test sample is placed on an ionization surface, and an intense electric field is used to generate analyte ions.

As used herein, the term “desorption” refers to the removal of an analyte from a surface and/or the entry of an analyte into a gaseous phase. Laser desorption thermal desorption is a technique wherein a sample containing the analyte is thermally desorbed into the gas phase by a laser pulse. The laser hits the back of a specially made 96-well plate with a metal base. The laser pulse heats the base and the heat causes the sample to transfer into the gas phase. The gas phase sample is then drawn into the mass spectrometer.

As used herein, the term “selective ion monitoring” is a detection mode for a mass spectrometric instrument in which only ions within a relatively narrow mass range, typically about one mass unit, are detected.

As used herein, “multiple reaction mode,” some-times known as “selected reaction monitoring,” is a detection mode for a mass spectrometric instrument in which a precursor ion and one or more fragment ions are selectively detected.

As used herein, the term “lower limit of quantification”, “lower limit of quantitation” or “LLOQ” refers to the point where measurements become quantitatively meaningful. The analyte response at this LOQ is identifiable, discrete and reproducible with a relative standard deviation (RSD %) of less than 20% and an accuracy of 85% to 115%.

As used herein, the term “limit of detection” or “LOD” is the point at which the measured value is larger than the uncertainty associated with it. The LOD is the point at which a value is beyond the uncertainty associated with its measurement and is defined as three times the RSD of the mean at the zero concentration.

As used herein, an “amount” of an analyte in a body fluid sample refers generally to an absolute value reflecting the mass of the analyte detectable in volume of sample. However, an amount also contemplates a relative amount in comparison to another analyte amount. For example, an amount of an analyte in a sample can be an amount which is greater than a control or normal level of the analyte normally present in the sample.

The term “about” as used herein in reference to quantitative measurements not including the measurement of the mass of an ion, refers to the indicated value plus or minus 10%. Mass spectrometry instruments can vary slightly in determining the mass of a given analyte. The term “about” in the context of the mass of an ion or the mass/charge ratio of an ion refers to +/−0.50 atomic mass unit.

As used herein, the term “amino acid” refers to natural amino acids, unnatural amino acids, and amino acid analogs, all in their D and L stereoisomers, unless otherwise indicated, if their structures allow such stereoisomeric forms.

As used herein, the term “peptide” refers a short polymer of amino acids linked together by peptide bonds. In contrast to other amino acid polymers (e.g., proteins, polypeptides, etc.), peptides are of, e.g., about 50 amino acids or less in length. A peptide may comprise natural amino acids, non-natural amino acids, amino acid analogs, and/or modified amino acids.

A peptide may be a subsequence of naturally occurring protein or a non-natural (synthetic) sequence.

As used herein, the term “mutant peptide” refers to a peptide having a distinct amino acid sequence from the most common variant occurring in nature, referred to as the “wild-type” sequence. A mutant peptide may be a subsequence of a mutant protein or polypeptide (e.g., a subsequence of a naturally-occurring protein that is not the most common sequence in nature) or may be a peptide that is not a subsequence of a naturally occurring protein or polypeptide. For example, a “mutant C-peptide” or a “C-peptide variant” may be a subsequence of a naturally-occurring, non-wild-type C-peptide, or may be distinct sequence not found in naturally-occurring C-peptide.

The summary above is non-limiting and other features and advantages of the technology will be apparent from the following detailed description, and from the claims.

### Methods

Insulin is a small peptide consisting of fifty-one amino acids. Insulin is composed of two chains, the α-chain and the β-chain, linked by disulfide bridges between cysteine residues. The α-chain has twenty-one amino acids and the β-chain has thirty amino acids.

Proinsulin is the prohormone precursor to insulin. Proinsulin metabolic intermediates may be formed during proinsulin processing, such as des-31,32-proinsulinor des-64,65-proinsulin. C-peptide is a peptide that connects insulin's 2 peptide chains and is released from proinsulin during processing and subsequently co-secreted from the pancreatic beta cell. Because of differences in half-life and hepatic clearance, peripheral blood levels of C-peptide and insulin are no longer equimolar but remain highly correlated. A mutant C-peptide (or a C-peptide variant) may be a subsequence of a naturally-occurring, non-wild-type C-peptide, or may be distinct sequence not found in naturally-occurring C-peptide. In some embodiments, a mutant C-peptide (or C-peptide variant) is C-peptide with one additional arginine residue on the C-terminal end (referred to as “C-peptide+R”). In some embodiments, a mutant C-peptide (or C-peptide variant) is C-peptide with two additional arginine residues on the C-terminal end (referred to as “C-peptide+RR”).

The determination of insulin in serum is primarily used for the diagnosis of glycemic disorders in diabetic and pre-diabetic patients in the assessment of insulin resistant syndromes. Accordingly, the methods described herein may be used for diagnosis of glycemic disorders or insulin resistant syndromes in diabetic and pre-diabetic patients. In some embodiments, the methods described herein may be used for diagnosing diabetes. In some embodiments, the methods described herein may be used for the assessment of insulin resistant syndromes. In some embodiments, the methods described herein may be used for distinguishing insulin-secreting tumors from exogenous insulin administration as a cause for hypoglycemia. In some embodiments, the methods described herein may be used for distinguishing type 1 diabetes from type 2 diabetes. In some embodiments, the methods described herein may be used for assessing the risk of diabetes in pre-diabetic patients.

In one aspect, provided herein are methods for measuring insulin levels in a patient by determining the amount of an analyte in a sample using mass spectrometry. As described herein, an analyte may comprise insulin, proinsulin, a proinsulin metabolic intermediate (e.g., des-31,32-proinsulin, des-64,65-proinsulin, or a mixture thereof), C-peptide, a mutant C-peptide (e.g., C-peptide+R, C-peptide+RR, or a mixture thereof), or a mixture of any two or more thereof. In some embodiments, the methods provided herein comprise multiplexed assays that simultaneously measure the amount of insulin, proinsulin, a metabolic intermediate of proinsulin, C-peptide, a mutant C-peptide, or a mixture of any two or more thereof in a sample by mass spectrometry. In some embodiments, the amount of an analyte in a sample is related to the amount of insulin in a patient. In some embodiments, the amount of, e.g., insulin and C-peptide, in a sample is used to determine the corresponding ratio (e.g., the ratio of insulin to C-peptide) in a patient.

In some embodiments, the methods described herein comprise: subjecting a sample to an enrichment process to obtain a fraction enriched in an analyte described herein; subjecting the enriched analyte to an ionization source under conditions suitable to generate one or more analyte ions detectable by mass spectrometry; determining the amount of one or more analyte ions by mass spectrometry. In some embodiments, the amount of the one or more analyte ions determined is used to determine the amount of the analyte in the sample. In some embodiments, the amount of the analyte in the sample is related to the amount of insulin in the patient.

In some embodiments, the methods described herein comprise subjecting a sample to an enrichment process to obtain a fraction enriched in analyte described herein. For example, the methods described herein may comprise immunocapturing the analyte from the sample. In some embodiments, immunocapturing provided herein comprises using antibodies (e.g., anti-insulin antibodies or anti-C-peptide antibodies). In some embodiments, the antibodies provided herein are monoclonal antibodies. In some embodiments, the antibodies provided herein are mouse monoclonal antibodies. In some embodiments, the antibodies provided herein are monoclonal IgG antibodies. In some embodiments, the antibodies provided herein are polyclonal antibodies. In some embodiments, the antibodies (e.g., anti-insulin antibodies or anti-C-peptide antibodies) are immobilized on magnetic beads. In some embodiments, the analyte immunocaptured on magnetic beads is washed and eluted.

In some embodiments, serum is delipidated prior to quantitation by mass spectrometry. In some embodiments, one or more delipidation reagents are used to remove lipids from the sample. In some embodiments, the delipidation reagent is CLEANASCITE®.

In some embodiments, the methods provided herein comprise purifying the samples prior to mass spectrometry. In some embodiments, the methods comprise purifying the samples using liquid chromatography. In some embodiments, liquid chromatography comprise high performance liquid chromatography (HPLC) or high turbulence liquid chromatograph (HTLC). In some embodiments, the methods comprise subjecting a sample to solid phase extraction (SPE).

In some embodiments, mass spectrometry comprises tandem mass spectrometry. In some embodiments, mass spectrometry is high resolution mass spectrometry. In some embodiments, mass spectrometry is high resolution/high accuracy mass spectrometry.

In some embodiments, ionization is by electrospray ionization (ESI). In some embodiments, ionization is by atmospheric pressure chemical ionization (APCI). In some embodiments, said ionization is in positive ion mode.

In some embodiments, methods provided herein comprise adding internal standards to the sample. In some embodiments, the internal standard for insulin is bovine insulin. In some embodiments, the internal standard for C-peptide is C-peptide heavy internal standard. In some embodiments, the internal standard is labeled. In some embodiments, the internal standard is deuterated or isotopically labeled.

In some embodiments, the patient sample is a serum sample. In some embodiments, the patient sample is a plasma sample. In some embodiments, the patient sample is a blood, saliva, or urine sample.

In some embodiments, the sample is subjected to acidic conditions prior to ionization. In some embodiments, subjecting the sample to acidic conditions comprises subjecting enriched analyte (e.g., an analyte described herein) to formic acid. In some embodiments, the sample is subjected to basic conditions prior to ionization. In some embodiments, subjecting the sample to basic conditions comprises subjecting the sample to trizma. In some embodiments, subjecting the sample to basic conditions comprises subjecting the sample to trizma and ethanol.

In some embodiments, provided herein is utilizing mass spectrometry for determining the amount of an analyte (e.g., an analyte described herein) in a sample, the methods include: (a) enriching the analyte in a sample by an extraction technique; (b) subjecting the purified analyte from step (a) to liquid chromatography to obtain a fraction enriched in the analyte from the sample; (c) subjecting the enriched analyte to an ionization source under conditions suitable to generate an analyte precursor ion detectable by mass spectrometry; (d) generating one or more fragment ions of the precursor ion; and (e) determining the amount of one or more of the fragment ions by mass spectrometry. In some embodiments, the amount of the one or more ions determined is used to determine the amount of the analyte in the sample.

In some embodiments, the collision energy is from about-90 eV to about-20 eV (e.g., from about-89 eV to about-29 eV). In some embodiments, the collision energy is from about 20 eV to about 50 eV (e.g., from about 27 eV to about 40 eV).

Thus, in accordance with some aspects, the present disclosure provides methods for determining an amount of an analyte (e.g., an analyte described herein) in a sample by mass spectrometry, the methods comprise one or more of: immunocapturing the analyte from the sample; subjecting the immunocaptured analyte to an ionization source under conditions suitable to generate one or more ions detectable by mass spectrometry; determining an amount of the one or more ions by mass spectrometry; and determining the amount of the analyte in the sample from the amount of the one or more ions.

In some embodiments, the analyte comprises insulin, proinsulin, a metabolic intermediate of proinsulin, C-peptide, a mutant C-peptide, or a mixture of any two or more thereof. In some embodiments, the analyte comprises or is proinsulin. In some embodiments, the analyte comprises or is a metabolic intermediate of proinsulin (e.g., des-31,32-proinsulin, des-64,65-proinsulin, or a mixture thereof). In some embodiments, the analyte comprises or is a mutant C-peptide (e.g., C-peptide+R, C-peptide+RR, or a mixture thereof). In some embodiments, the analyte is not insulin (e.g., the analyte may comprise insulin, but further comprises at least one additional species, such as a species selected from proinsulin, a proinsulin metabolic intermediate, and a mutant C-peptide). In some embodiments, the analyte is not C-peptide (e.g., the analyte may comprise C-peptide, but further comprises at least one additional species, such as a species selected from proinsulin, a proinsulin metabolic intermediate, and a mutant C-peptide). In some embodiments, the is not a mixture of insulin and C-peptide (e.g., the analyte may comprise a mixture of insulin and C-peptide, but further comprises at least one additional species, such as a species selected from proinsulin, a proinsulin metabolic intermediate, and a mutant C-peptide).

In some embodiments, the present disclosure provides methods for determining an amount of an analyte (e.g., an analyte described herein) in a sample by mass spectrometry, the methods comprise one or more of: immunocapturing the analyte from the sample; subjecting the immunocaptured analyte to an ionization source under conditions suitable to generate one or more ions detectable by mass spectrometry; determining an amount of the one or more ions by mass spectrometry; and determining the amount of the analyte in the sample from the amount of the one or more ions; wherein the analyte comprises insulin, proinsulin, a metabolic intermediate of proinsulin, C-peptide, a mutant C-peptide, or a mixture of any two or more thereof; the metabolic intermediate of proinsulin is des-31,32-proinsulin, des-64,65-proinsulin, or a mixture thereof; and the mutant C-peptide is C-peptide+R, C-peptide+RR, or a mixture thereof.

In some embodiments, the present disclosure provides methods for determining an amount of an analyte (e.g., an analyte described herein) in a sample by mass spectrometry, the methods comprise one or more of: immunocapturing the analyte from the sample; subjecting the immunocaptured analyte to an ionization source under conditions suitable to generate one or more ions detectable by mass spectrometry; determining an amount of the one or more ions by mass spectrometry; and determining the amount of the analyte in the sample from the amount of the one or more ions; wherein the analyte comprises insulin, proinsulin, a metabolic intermediate of proinsulin, C-peptide, a mutant C-peptide, or a mixture of any two or more thereof; the metabolic intermediate of proinsulin is des-31,32-proinsulin, des-64,65-proinsulin, or a mixture thereof; the mutant C-peptide is C-peptide+R, C-peptide+RR, or a mixture thereof; and the analyte is not insulin, C-peptide, and/or a mixture of insulin and C-peptide.

In some embodiments, the analyte described herein comprises or is proinsulin. Accordingly, the present disclosure provides methods for determining an amount of proinsulin in a sample, the methods comprise one or more of: immunocapturing proinsulin from the sample; subjecting the immunocaptured proinsulin to an ionization source under conditions suitable to generate one or more proinsulin ions detectable by mass spectrometry; determining an amount of the one or more proinsulin ions by mass spectrometry; and determining the amount of proinsulin in the sample from the amount of the one or more proinsulin ions.

In some embodiments, the analyte described herein comprises or is a metabolic intermediate of proinsulin (e.g., des-31,32-proinsulin, des-64,65-proinsulin, or a mixture thereof). Accordingly, the present disclosure provides methods for determining an amount of a metabolic intermediate of proinsulin in a sample, the methods comprise one or more of: immunocapturing the metabolic intermediate from the sample; subjecting the immunocaptured metabolic intermediate to an ionization source under conditions suitable to generate one or more metabolic intermediate ions detectable by mass spectrometry; determining an amount of the one or more metabolic intermediate ions by mass spectrometry; and determining the amount of the metabolic intermediate of proinsulin in the sample from the amount of the one or more metabolic intermediate ions.

In some embodiments, the analyte described herein comprises or is a mutant C-peptide (e.g., C-peptide+R, C-peptide+RR, or a mixture thereof). Accordingly, the present disclosure provides methods for determining an amount of a mutant C-peptide in a sample, the methods comprise one or more of: immunocapturing the mutant C-peptide from the sample; subjecting the immunocaptured mutant C-peptide to an ionization source under conditions suitable to generate one or more mutant C-peptide ions detectable by mass spectrometry; determining an amount of the one or more mutant C-peptide ions by mass spectrometry; and determining the amount of the mutant C-peptide in the sample from the amount of the one or more mutant C-peptide ions;

Suitable test samples for use in methods of the present invention include any test sample that may contain the analyte of interest. In some preferred embodiments, a sample is a biological sample; that is, a sample obtained from any biological source, such as an animal, a cell culture, an organ culture, etc. In certain preferred embodiments, samples are obtained from a mammalian animal, such as a dog, cat, horse, etc. Particularly preferred mammalian animals are primates, most preferably male or female humans. Preferred samples comprise bodily fluids such as blood, plasma, serum, saliva, cerebrospinal fluid, or tissue samples; preferably plasma and serum. Such samples may be obtained, for example, from a patient; that is, a living person, male or female, presenting oneself in a clinical setting for diagnosis, prognosis, or treatment of a disease or condition. In embodiments where the sample comprises a biological sample, the methods may be used to determine the amount of an analyte in the sample when the sample was obtained from the biological source.

The present invention also contemplates kits for a quantitation assay (e.g., a quantitation assay for determining the amount of an analyte described herein, such as insulin, proinsulin, a proinsulin metabolic intermediate (e.g., des-31,32-proinsulin, des-64,65-proinsulin, or a mixture thereof), C-peptide, a mutant C-peptide (e.g., C-peptide+R, C-peptide+RR, or a mixture thereof), or a mixture of any two or more thereof. A kit for a quantitation assay may include a kit comprising the compositions provided herein. For example, a kit may include packaging material and measured amounts of an isotopically labeled internal standard, in amounts sufficient for at least one assay. Typically, the kits will also include instructions recorded in a tangible form (e.g., contained on paper or an electronic medium) for using the packaged reagents for use in a quantitation assay.

Calibration and QC pools for use in embodiments of the present invention are preferably prepared using a matrix similar to the intended sample matrix, provided that insulin is essentially absent.

### Sample Preparation for Mass Spectrometric Analysis

In preparation for mass spectrometric analysis, the analyte described herein may be enriched relative to one or more other components in the sample by various methods known in the art, including for example, immunocapture, liquid chromatography, filtration, centrifugation, thin layer chromatography (TLC), electrophoresis including capillary electrophoresis, affinity separations including immunoaffinity separations, extraction methods including ethyl acetate or methanol extraction, and the use of chaotropic agents or any combination of the above or the like.

One method of sample purification that may be used prior to mass spectrometry is applying a sample to a solid-phase extraction (SPE) column under conditions where the analyte of interest is reversibly retained by the column packing material, while one or more other materials are not retained. In this technique, a first mobile phase condition can be employed where the analyte of interest is retained by the column, and a second mobile phase condition can subsequently be employed to remove retained material from the column, once the non-retained materials are washed through.

In some embodiments, the analyte in a sample may be reversibly retained on a SPE column with a packing material comprising an alkyl bonded surface. For example, in some embodiments, a C-8 on-line SPE column (such as an Oasis HLB on-line SPE column/cartridge (2.1 mm×20 mm) from Phenomenex, Inc. or equivalent) may be used to enrich insulin prior to mass spectrometric analysis. In some embodiments, use of an SPE column is conducted with HPLC Grade 0.2% aqueous formic acid as a wash solution, and use of 0.2% formic acid in acetonitrile as an elution solution.

In other embodiments, the methods include immunopurifying the analyte prior to mass spectrometry analysis. The immunopurification step may be performed using any of the immunopurification methods well known in the art. Often the immunopurification procedure utilizes antibodies bound, conjugated, immobilized or otherwise attached to a solid support, for example a column, well, tube, capsule, particle or the like. Generally, immunopurification methods involve (1) incubating a sample containing the analyte of interest with antibodies such that the analyte binds to the antibodies, (2) performing one or more washing steps, and (3) eluting the analyte from the antibodies.

In certain embodiments the incubation step of the immunopurification is performed with the antibodies free in solution and the antibodies are subsequently bound or attached to a solid surface prior to the washing steps. In certain embodiments this can be achieved using a primary antibody (e.g. an anti-insulin antibody and/or an anti-C-peptide antibody) and a secondary antibody attached to a solid surface that has an affinity to the primary anti-insulin antibody. In alternative embodiments, the primary antibody is bound to the solid surface prior to the incubation step.

Appropriate solid supports include without limitation tubes, slides, columns, beads, capsules, particles, gels, and the like. In some preferred embodiments, the solid support is a multi-well plate, such as, for example, a 96 well plate, a 384-well plate or the like. In some embodiments the solid support are sepharose or agarose beads or gels. There are numerous methods well known in the art by which antibodies (for example, an insulin antibody or a secondary antibody) may be bound, attached, immobilized or coupled to a solid support, e.g., covalent or non-covalent linkages adsorption, affinity binding, ionic linkages and the like. In some embodiments antibodies are coupled using CNBr, for example the antibodies may be coupled to CNBr activated sepharose. In other embodiments, the antibody is attached to the solid support through an antibody binding protein such as protein A, protein G, protein A/G, or protein L.

The washing step of the immunopurification methods generally involve washing the solid support such that the analyte remain bound to the corresponding antibodies on the solid support. The elution step of the immunopurification generally involves the addition of a solution that disrupts the binding of the analyte to the corresponding antibodies. Exemplary elution solutions include organic solutions, salt solutions, and high or low pH solutions.

Another method of sample purification that may be used prior to mass spectrometry is liquid chromatography (LC). In liquid chromatography techniques, an analyte may be purified by applying a sample to a chromatographic analytical column under mobile phase conditions where the analyte of interest elutes at a differential rate in comparison to one or more other materials. Such procedures may enrich the amount of one or more analytes of interest relative to one or more other components of the sample.

Certain methods of liquid chromatography, including HPLC, rely on relatively slow, laminar flow technology. Traditional HPLC analysis relies on column packing in which laminar flow of the sample through the column is the basis for separation of the analyte of interest from the sample. The skilled artisan will understand that separation in such columns is a partition process and may select LC, including HPLC, instruments and columns that are suitable for use with C peptide. The chromatographic analytical column typically includes a medium (i.e., a packing material) to facilitate separation of chemical moieties (i.e., fractionation). The medium may include minute particles. The particles typically include a bonded surface that interacts with the various chemical moieties to facilitate separation of the chemical moieties. One suitable bonded surface is a hydrophobic bonded surface such as an alkyl bonded or a cyano bonded surface. Alkyl bonded surfaces may include C-4, C-8, C-12, or C-18 bonded alkyl groups. In some embodiments, the chromatographic analytical column is a monolithic C-18 column. The chromatographic analytical column includes an inlet port for receiving a sample and an outlet port for discharging an effluent that includes the fractionated sample. The sample may be supplied to the inlet port directly, or from a SPE column, such as an on-line SPE column or a TFLC column. In some embodiments, an on-line filter may be used ahead of the SPE column and or HPLC column to remove particulates and phospholipids in the samples prior to the samples reaching the SPE and/or TFLC and/or HPLC columns.

In one embodiment, the sample may be applied to the LC column at the inlet port, eluted with a solvent or solvent mixture, and discharged at the outlet port. Different solvent modes may be selected for eluting the analyte(s) of interest. For example, liquid chromatography may be performed using a gradient mode, an isocratic mode, or a polytypic (i.e. mixed) mode. During chromatography, the separation of materials is effected by variables such as choice of eluent (also known as a “mobile phase”), elution mode, gradient conditions, temperature, etc.

In some embodiments, the analyte in a sample is enriched with HPLC. This HPLC may be conducted with a monolithic C-18 column chromatographic system, for example, an Onyx Monolithic C-18 column from Phenomenex Inc. (50×2.0 mm), or equivalent. In certain embodiments, HPLC is performed using HPLC Grade 0.2% aqueous formic acid as solvent A, and 0.2% formic acid in acetonitrile as solvent B.

By careful selection of valves and connector plumbing, two or more chromatography columns may be connected as needed such that material is passed from one to the next without the need for any manual steps. In preferred embodiments, the selection of valves and plumbing is controlled by a computer pre-programmed to perform the necessary steps. Most preferably, the chromatography system is also connected in such an on-line fashion to the detector system, e.g., an MS system. Thus, an operator may place a tray of samples in an autosampler, and the remaining operations are performed under computer control, resulting in purification and analysis of all samples selected.

In some embodiments, TFLC may be used for purification of the analyte prior to mass spectrometry. In such embodiments, samples may be extracted using a TFLC column which captures the analyte. The analyte is then eluted and transferred on-line to an analytical HPLC column. For example, sample extraction may be accomplished with a TFLC extraction cartridge with a large particle size (50 μm) packing. Sample eluted off of this column may then be transferred on-line to an HPLC analytical column for further purification prior to mass spectrometry. Because the steps involved in these chromatography procedures may be linked in an automated fashion, the requirement for operator involvement during the purification of the analyte can be minimized. This feature may result in savings of time and costs, and eliminate the opportunity for operator error.

In some embodiments, one or more of the above purification techniques may be used in parallel for purification of the analyte to allow for simultaneous processing of multiple samples. In some embodiments, the purification techniques employed exclude immunopurification techniques, such as immunoaflinity chromatography.

### Detection and Quantitation by Mass Spectrometry

Mass spectrometry is performed using a mass spectrometer, which includes an ion source for ionizing the fractionated sample and creating charged molecules for further analysis. In various embodiments, the analyte may be ionized by any method known to the skilled artisan. For example, ionization of an analyte may be performed by electron ionization, chemical ionization, electrospray ionization (ESI), photon ionization, atmospheric pressure chemical ionization (APCI), photoionization, atmospheric pressure photoionization (APPI), laser diode thermal desorption (LDTD), fast atom bombardment (FAB), liquid secondary ionization (LSI), matrix assisted laser desorption ionization (MALDI), field ionization, field desorption, thermospray/plasmaspray ionization, surface enhanced laser desorption ionization (SELDI), inductively coupled plasma (ICP) and particle beam ionization. The skilled artisan will understand that the choice of ionization method may be determined based on the analyte to be measured, type of sample, the type of detector, the choice of positive versus negative mode, etc. The analyte may be ionized in positive or negative mode. In preferred embodiments, the analyte is ionized by ESI in positive ion mode.

In mass spectrometry techniques generally, after the sample has been ionized, the positively or negatively charged ions thereby created may be analyzed to determine a mass to charge ratio (m/z). Various analyzers for determining m/z include quadrupole analyzers, ion traps analyzers, time-of-flight analyzers, Fourier transform ion cyclotron resonance mass analyzers, and orbitrap analyzers. Some exemplary ion trap methods are described in Bartolucci, et al., Rapid Commun. Mass Spectrom. 2000, 14:967-73.

The ions may be detected using several detection modes. For example, selected ions may be detected, i.e. using a selective ion monitoring mode (SIM), or alternatively, mass transitions resulting from collision induced dissociation or neutral loss may be monitored, e.g., multiple reaction monitoring (MRM) or selected reaction monitoring (SRM). In some embodiments, the mass-to-charge ratio is determined using a quadrupole analyzer. For example, in a “quadrupole” or “quadrupole ion trap” instrument, ions in an oscillating radio frequency field experience a force proportional to the DC potential applied between electrodes, the amplitude of the RF signal, and the mass/charge ratio. The voltage and amplitude may be selected so that only ions having a particular mass/charge ratio travel the length of the quadrupole, while all other ions are deflected. Thus, quadrupole instruments may act as both a “mass filter” and as a “mass detector” for the ions injected into the instrument.

One may enhance the resolution of MS techniques employing certain mass spectrometric analyzers through “tandem mass spectrometry,” or “MS/MS”. In this technique, a precursor ion (also called a parent ion) generated from a molecule of interest can be filtered in an MS instrument, and the precursor ion subsequently fragmented to yield one or more fragment ions (also called daughter ions or product ions) that are then analyzed in a second MS procedure. By careful selection of precursor ions, only ions produced by certain analytes are passed to the fragmentation chamber, where collisions with atoms of an inert gas produce the fragment ions. Because both the precursor and fragment ions are produced in a reproducible fashion under a given set of ionization/fragmentation conditions, the MS/MS technique may provide an extremely powerful analytical tool. For example, the combination of filtration/fragmentation may be used to eliminate interfering substances, and may be particularly useful in complex samples, such as biological samples. In certain embodiments, a mass spectrometric instrument with multiple quadrupole analyzers (such as a triple quadrupole instrument) is employed to conduct tandem mass spectrometric analysis.

In certain embodiments, such as MS/MS, where precursor ions are isolated for further fragmentation, collision activation dissociation is often used to generate the fragment ions for further detection. In collision activated dissociation (CAD), precursor ions in energy through collisions with an inert gas, and subsequently fragment by a process referred to as “unimolecular decomposition.” Sufficient energy must be deposited in the precursor ion so that certain bonds within the ion can be broken due to increased vibrational energy.

In some embodiments, the analyte in a sample is detected and/or quantified using MS/MS as follows. The analyte is enriched in a sample by first subjecting the sample to SPE, then to liquid chromatography, preferably HPLC; the flow of liquid solvent from a chromatographic analytical column enters the heated nebulizer interface of an MS/MS analyzer; and the solvent/analyte mixture is converted to vapor in the heated charged tubing of the interface. During these processes, the analyte is ionized. The ions, e.g. precursor ions, pass through the orifice of the instrument and enter the first quadrupole. Quadrupoles 1 and 3 (Q1 and Q3) are mass filters, allowing selection of ions (i.e., selection of “precursor” and “fragment” ions in QI and Q3, respectively) based on their mass to charge ratio (m/z). Quadrupole 2 (Q2) is the collision cell, where ions are fragmented. The first quadrupole of the mass spectrometer (Q1) selects for molecules with the m/z of an analyte ion. Precursor ions with the correct m/z are allowed to pass into the collision chamber (Q2), while unwanted ions with any other m/z collide with the sides of the quadrupole and are eliminated. Precursor ions entering Q2 collide with neutral gas molecules (such as Argon molecules) and fragment. The fragment ions generated are passed into quadrupole 3 (Q3), where the fragment ions are selected for detection.

The methods may involve MS/MS performed in either positive or negative ion mode; preferably positive ion mode. Using standard methods well known in the art, one of ordinary skill is capable of identifying one or more fragment ions of a particular precursor ion of an analyte described herein that may be used for selection in quadrupole 3 (Q3). In certain embodiments, the relative abundance of a single fragment ion from a single precursor ion may be measured. Alternatively, the relative abundances of two or more fragment ions from a single precursor ion may be measured. In these embodiments, the relative abundances of each fragment ion may be subjected to any known mathematical treatment to quantitatively assess the corresponding analyte originally in the sample. In other embodiments, one or more fragment ions from two or more precursor ions may be measured and utilized as above to qualitatively assess insulin originally in the sample.

As ions collide with the detector they produce a pulse of electrons that are converted to a digital signal. The acquired data is relayed to a computer, which plots counts of the ions collected versus time. The resulting mass chromatograms are similar to chromatograms generated in traditional HPLC methods. The areas under the peaks corresponding to particular ions, or the amplitude of such peaks, are measured and the area or amplitude is correlated to the amount of the analyte of interest. In certain embodiments, the area under the curves, or amplitude of the peaks, for fragment ion(s) and/or precursor ions are measured to determine the amount of analyte or analytes detected. As described above, the relative abundance of a given ion or parent/fragment ion pair may be converted into an absolute amount of an original analyte (e.g., an analyte described herein), using calibration standard curves based on peaks of one or more ions of an internal molecular standard (e.g., an internal molecular standard described herein).

Ionization of an analyte may result in multiply charged precursor ions (such as precursor ions of 4+, 5+, 6+, etc.). In some embodiments, an analyte may comprise insulin. Accordingly, ionization conditions, particularly the pH of the buffer utilized in electrospray techniques, greatly influence the identity and quantity of insulin precursor ions generated. For example, under acidic conditions, positive electrospray ionization may predominately generate 5+ and 6+ charged insulin precursor ions. However, under basic conditions, positive electrospray ionization may predominately generate 4+ and 5+ charged insulin precursor ions. The methods may utilize either acidic or basic conditions; preferably acidic conditions.

Alternate modes of operating a tandem mass spectrometric instrument that may be used in certain embodiments include product ion scanning and precursor ion scanning. For a description of these modes of operation, see, e.g., E. Michael Thurman, et al., Chromatographic-Mass Spectrometric Food Analysis for Trace Determination of Pesticide Residues, Chapter 8 (Amadeo R. Fernandez-Alba, ed., Elsevier 2005) (387).

In other embodiments, a high resolution/high accuracy mass analyzer may be used for quantitative analysis of an analyte described herein in accordance with the methods of the present invention. In some embodiments, to achieve acceptable precision for quantitative results, the mass spectrometer is capable of exhibiting a resolving power (FWHM) of 10,000 or more, with accuracy of about 50 ppm or less for the ions of interest. In some embodiments, the mass spectrometer exhibits a resolving power (FWHM) of 18,000 or better, with accuracy of about 5 ppm or less; such as a resolving power (FWHM) of 20,000 or better and accuracy of about 3 ppm or less; such as a resolving power (FWHM) of 25,000 or better and accuracy of about 3 ppm or less. Exemplary analyzers capable of exhibiting the requisite level of performance include orbitrap mass analyzers, certain TOF mass analyzers, and Fourier transform ion cyclotron resonance mass analyzers.

Elements found in biological active molecules, such as carbon, oxygen, and nitrogen, naturally exist in a number of different isotopic forms. For example, most carbon is present as ¹²C, but approximately 1% of all naturally occurring carbon is present as ¹³C. Thus, some fraction of naturally occurring molecules containing at least one carbon atom will contain at least one ¹³C atom. Inclusion of naturally occurring elemental isotopes in molecules gives rise to multiple molecular isotopic forms. The difference in masses of molecular isotopic forms is at least 1 atomic mass unit (amu). This is because elemental isotopes differ by at least one neutron (mass of one neutron≈1 amu). When molecular isotopic forms are ionized to multiply charged states, the mass distinction between the isotopic forms can become difficult to discern because mass spectrometric detection is based on the mass to charge ratio (m/z). For example, two isotopic forms differing in mass by 1 amu that are both ionized to a 5+ state will exhibit differences in their m/z of only 0.2. High resolution/high accuracy mass spectrometers are capable of discerning between isotopic forms of highly multiply charged ions (such as ions with charges of ±2, ±3, ±4, ±5, or higher).

Due to naturally occurring elemental isotopes, multiple isotopic forms typically exist for every molecular ion (each of which may give rise to a separately detectable spectrometric peak if analyzed with a sensitive enough mass spectrometric instrument). The m/z ratios and relative abundances of multiple isotopic forms collectively comprise an isotopic signature for a molecular ion. In some embodiments, the m/z ratios and relative abundances for two or more molecular isotopic forms may be utilized to confirm the identity of a molecular ion under investigation. In some embodiments, the mass spectrometric peak from one or more isotopic forms is used to quantitate a molecular ion. In some related embodiments, a single mass spectrometric peak from one isotopic form is used to quantitate a molecular ion. In other related embodiments, a plurality of isotopic peaks are used to quantitate a molecular ion. In these later embodiments, the plurality of isotopic peaks may be subject to any appropriate mathematical treatment. Several mathematical treatments are known in the art and include, but are not limited to summing the area under multiple peaks, or averaging the response from multiple peaks.

In some embodiments, the relative abundance of one or more ion is measured with a high resolution/high accuracy mass spectrometer in order to qualitatively assess the amount of an analyte (e.g., an analyte described herein) in the sample. In some embodiments, the one or more ions measured by high resolution/high accuracy mass spectrometry are multiply charged analyte ions.

Use of high resolution orbitrap analyzers has been reported for qualitative and quantitative analyses of various analytes. See, e.g., U.S. Patent Application Pub. No. 2008/0118932 (filed Nov. 9, 2007); Bredehoft, et al., Rapid Commun. Mass Spectrom., 2008, 22:477-485; Le Breton, et al., Rapid Commun. Mass Spectrom., 2008, 22:3130-36; Thevis, et al., Mass Spectrom. Reviews, 2008, 27:35-50; Thomas, et al., J. Mass Spectrom., 2008, 43:908-15; Schenk, et al., BMC Medical Genomics, 2008, 1:41; and Olsen, et al., Nature Methods, 2007, 4:709-12.

The results of an analyte assay may be related to the amount of the analyte in the original sample by numerous methods known in the art. For example, given that sampling and analysis parameters are carefully controlled, the relative abundance of a given ion may be compared to a table that converts that relative abundance to an absolute amount of the original molecule. Alternatively, external standards may be run with the samples, and a standard curve constructed based on ions generated from those standards. Using such a standard curve, the relative abundance of a given ion may be converted into an absolute amount of the original molecule. In certain preferred embodiments, an internal standard is used to generate a standard curve for calculating the quantity of insulin. Methods of generating and using such standard curves are well known in the art and one of ordinary skill is capable of selecting an appropriate internal standard. For example, one or more forms of isotopically labeled molecules may be used as internal standards. Numerous other methods for relating the amount of an ion to the amount of the original molecule will be well known to those of ordinary skill in the art.

As used herein, an “isotopic label” produces a mass shift in the labeled molecule relative to the unlabeled molecule when analyzed by mass spectrometric techniques. Examples of suitable labels include deuterium (²H), ¹³C, and ¹⁵N. One or more isotopic labels can be incorporated at one or more positions in the molecule and one or more kinds of isotopic labels can be used on the same isotopically labeled molecule.

In other embodiments, insulin may be subjected to a chemical treatment to generate insulin's constituent chains prior to mass spectrometric analysis. Insulin's β-chain may be separated by any chemical treatment known in the art to cause disulfide reduction. For example, insulin may be treated with TCEP (tris(2-carboxyethyl) phosphine to reduce insulin's disulfide bridges and separate the α-chain and β-chain.

The β-chains may then be subject to any one or more of the purification steps described above for purification of insulin. In preferred embodiments, β-chains are subject to purification by HPLC prior to mass spectrometric analysis.

Once purified, β-chains are then subjected to an ionization source. As with insulin, the skilled artisan will understand that the choice of ionization method may be determined based on the analyte to be measured, type of sample, the type of detector, the choice of positive versus negative mode, etc. Insulin β-chains may be ionized in positive or negative mode. In preferred embodiments, insulin β-chains are ionized by ESI in positive mode.

Ionization of insulin β-chains may result in multiply charged β-chain precursor ions (such as precursor ions of 3+, 4+, 5+, etc.). Similar to insulin, the identity and quantity of the multiply charged species generated from ionization of insulin β-chains is affected by the ionization conditions employed. In preferred embodiments, insulin β-chains are ionized under acidic conditions.

One or more steps of any of the above described methods may be performed using automated machines. In certain embodiments, one or more purification steps are performed on-line, and more preferably all of the purification and mass spectrometry steps may be performed in an on-line fashion.

In some embodiments, the analyte comprises or is proinsulin. Ionization of proinsulin may result in multiply charged proinsulin precursor ions (such as precursor ions of 3+, 4+, 5+, etc.). For example, positive electrospray ionization of proinsulin molecules may generate 7+ charged proinsulin precursor ions with m/z of about 1342.20±0.5. Fragmentation of this proinsulin precursor ion may generate fragment ions with m/z of about 183.30±0.5, 120.20±0.5, and 219.30±0.5.

In some embodiments, the analyte comprises or is des-31,32-proinsulin. Ionization of des-31,32-proinsulin may result in multiply charged des-31,32-proinsulin precursor ions (such as precursor ions of 3+, 4+, 5+, etc.). For example, positive electrospray ionization of des-31,32-proinsulin molecules may generate 7+ charged des-31,32-proinsulin precursor ions with m/z of about 1299.50±0.5. Fragmentation of this des-31,32-proinsulin precursor ion may generate des-31,32-proinsulin fragment ions with m/z of about 129.20±0.5, 226.20±0.5, and 183.25±0.5.

In some embodiments, the analyte comprises or is des-64,65-proinsulin. Ionization of des-64,65-proinsulin may result in multiply charged des-64,65-proinsulin precursor ions (such as precursor ions of 3+, 4+, 5+, etc.). For example, positive electrospray ionization of des-64,65-proinsulin molecules may generate 7+ charged des-64,65-proinsulin precursor ions with m/z of about 1304.30±0.5. Fragmentation of this des-64,65-proinsulin precursor ion may generate des-64,65-proinsulin fragment ions with m/z of about 147.15±0.5, 130.15±0.5, and 183.20±0.5.

In some embodiments, the analyte comprises or is C-peptide+R. Ionization of C-peptide+R may result in multiply charged C-peptide+R precursor ions (such as precursor ions of 3+, 4+, 5+, etc.). For example, positive electrospray ionization of C-peptide+R molecules may generate 3+ charged C-peptide+R precursor ions with m/z of about 1059.20±0.5. Fragmentation of this C-peptide+R precursor ion may generate C-peptide+R fragment ions with m/z of about 966.40±0.5, 260.00±0.5, and 844.30=0.5.

In some embodiments, the analyte comprises or is C-peptide+RR. Ionization of C-peptide+RR may result in multiply charged C-peptide+RR precursor ions (such as precursor ions of 3+, 4+, 5+, etc.). For example, positive electrospray ionization of C-peptide+RR molecules may generate 3+ charged C-peptide+RR precursor ions with m/z of about 1111.50±0.5. Fragmentation of this C-peptide+RR precursor ion may generate C-peptide+RR fragment ions with m/z of about 896.50±0.5, 499.90±0.5, and 260.30±0.5.

In some embodiments, the analyte comprises insulin but is not insulin (e.g., the analyte further comprises at least one additional species, such as a species selected from proinsulin, a proinsulin metabolic intermediate, and a mutant C-peptide). Ionization of insulin may result in multiply charged insulin precursor ions (such as precursor ions of 3+, 4+, 5+, etc.). For example, positive electrospray ionization of insulin molecules may generate 5+ charged insulin precursor ions with m/z of about 1162.30±0.5. Fragmentation of this insulin precursor ion may generate insulin fragment ions with m/z of about 226.20±0.5, 136.10±0.5, and 345.20±0.5.

In some embodiments, the analyte comprises C-peptide but is not C-peptide (e.g., the analyte further comprises at least one additional species, such as a species selected from proinsulin, a proinsulin metabolic intermediate, and a mutant C-peptide). Ionization of C-peptide may result in multiply charged C-peptide precursor ions (such as precursor ions of 3+, 4+, 5+, etc.). For example, positive electrospray ionization of C-peptide molecules may generate 3+ charged C-peptide precursor ions with m/z of about 1007.70±0.5. Fragmentation of this C-peptide precursor ion may generate C-peptide fragment ions with m/z of about 927.50±0.5, 646.40±0.5, and 533.30±0.5.

In some embodiments, the limit of quantitation of a method described herein (e.g., a method for determining an amount of insulin) is less than or equal to about 3 ulU/mL. In some embodiments, the limit of quantitation of a method described herein (e.g., a method for determining an amount of C-peptide or a mutant C-peptide) is less than or equal to about 0.2 ng/ml (e.g., less than or equal to about 0.11 ng/mL). In some embodiments, the limit of quantitation of a method described herein (e.g., a method for determining an amount of proinsulin or a metabolic intermediate of proinsulin) is less than or equal to about 3 pmol/L (e.g., less than or equal to about 2.9 pmol/L).

In some embodiments, the amounts of two or more or all of: insulin, proinsulin, a proinsulin metabolic intermediate (e.g., des-31,32-proinsulin, des-64,65-proinsulin, or a mixture thereof), C-peptide, and a mutant C-peptide (e.g., C-peptide+R, C-peptide+RR, or a mixture thereof) are determined in the same sample injection. In some embodiments, the amounts of insulin, proinsulin, des-31,32-proinsulin, des-64,65-proinsulin, C-peptide, C-peptide+R, and C-peptide+RR are determined in the same sample injection.

The present invention, thus generally described, will be understood more readily by reference to the following examples, which are provided by way of illustration and are not intended to be limiting of the present invention.

### EXAMPLES

### Example 1

The insulin, C-peptide, proinsulin, des-31,32-proinsulin, des-64,65-proinsulin, C-peptide+R, and/or C-peptide+RR in a serum sample can be detected and quantified in accordance with methods summarized below.

In this assay, serum was first delipidated, and then the analytes (e.g., insulin, C-peptide, proinsulin, des-31,32-proinsulin, des-64,65-proinsulin, C-peptide+R, and/or C-peptide+RR) were immunocaptured (immunoprecipitated, IP) using antibodies immobilized on magnetic beads. The beads were subjected to a rigorous washing regime to remove non-specifically bound material and the peptides were subsequently eluted from the beads with acidified acetonitrile in water. An aliquot of Trizma base was added to enhance stability of the peptides in the elution plate.

The processes of calibrator preparation, internal standard addition, delipidation, bead deposition, immunocapture, washing and eluting the peptides from the beads were automated, using a TECAN Evo liquid handler. Manual steps involved initial dilution of calibrator stocks, transfer to the centrifuge post-delipidation and bead preparation.

The elution plate was transferred from the deck of the TECAN robot to the autosampler of a Shimadzu QX (Nexera XR) system and immediately run. The sample was injected onto a hydrophilic/lipophilic balanced (HLB) capture column where the analytes were further enriched from background contaminants. After washing, a plug of transfer solvent was used to liberate the peptides from the extraction cartridge and transfer them to a reversed phase analytical column. An acetonitrile gradient chromatographically resolved the analytes from the remaining background contaminants and each other.

The flow of solvent from the HPLC column was directed to the heated electrospray source of a Shimadzu 8060NX mass spectrometer. In the mass spectrometer, only the ions with the desired mass to charge ratio were allowed to pass through the Quadrupole 1 (Q1) area into the collision chamber (Q2). Then the accelerated ions collide with neutral argon gas molecules to become small fragments. Finally, in Q3 only the selected ions were chosen to reach the detector (see Table 1 below). The intensity of the signal at the detector was proportional to the number of molecules entering the mass spectrometer. Peak area ratios were then calculated for a set of known calibrators and calibration curves were established. The calibration equation can then be used to determine the concentration of the analytes (e.g., insulin, C-peptide, proinsulin, des-31,32-proinsulin, des-64,65-proinsulin, C-peptide+R, and/or C-peptide+RR) in the sample.

TABLE 1

| Analyte | Charge State | Parent Ion m/z | Product Ions m/z |
|---|---|---|---|
| Insulin | +5 | 1162.30 | 226.20, 136.10, 345.20 |
| Bovine Insulin | +5 | 1147.40 | 136.10, 226.20, 315.10 |
| C-peptide | +3 | 1007.70 | 927.50, 646.40, 533.30 |
| C-peptide ISTD | +3 | 1009.50 | 934.50, 653.40, 540.30 |
| Proinsulin | +7 | 1342.20 | 183.30, 120.20, 219.30 |
| Des-31,32- | +7 | 1299.50 | 129.20, 226.20, 183.25 |
| proinsulin | | | |
| Des-64,65- | +7 | 1304.30 | 147.15, 130.15, 183.20 |
| proinsulin | | | |
| C-peptide + R | +3 | 1059.20 | 966.40, 260.00, 844.30 |
| C-peptide + RR | +3 | 1111.50 | 896.50, 499.90, 260.30 |

Para. 1. A method for determining an amount of an analyte in a sample by mass spectrometry, the method comprising: immunocapturing the analyte from the sample; subjecting the immunocaptured analyte to an ionization source under conditions suitable to generate one or more ions detectable by mass spectrometry; determining an amount of the one or more ions by mass spectrometry; and determining the amount of the analyte in the sample from the amount of the one or more ions; wherein the analyte comprises insulin, proinsulin, a metabolic intermediate of proinsulin, C-peptide, a mutant C-peptide, or a mixture of any two or more thereof, the metabolic intermediate of proinsulin is des-31,32-proinsulin, des-64,65-proinsulin, or a mixture thereof; and the mutant C-peptide is C-peptide with one additional arginine residue on C-terminal end (C-peptide+R), C-peptide with two additional arginine residues on C-terminal end (C-peptide+RR), or a mixture thereof.

Para. 2. The method of Para. 1, wherein the analyte comprises proinsulin.

Para. 3. The method of Para. 1 or 2, wherein the one or more ions comprise one or more proinsulin ions.

Para. 4. The method of any one of Paras. 1-3, wherein the one or more proinsulin ions comprise a proinsulin precursor ion with a mass-to-charge ratio (m/z) of 1342.20±0.5.

Para. 5. The method of Para. 3, wherein the one or more proinsulin ions comprise one or more proinsulin fragment ions selected from a group consisting of ions with m/z of 183.30±0.5, 120.20±0.5, and 219.30±0.5.

Para. 6. The method of any one of Paras. 1-5, wherein the analyte comprises a metabolic intermediate of proinsulin.

Para. 7. The method of Para. 6, wherein the metabolic intermediate of proinsulin comprises des-31,32-proinsulin.

Para. 8. The method of Para. 7, wherein the one or more ions comprise one or more des-31,32-proinsulin ions.

Para. 9. The method of Para. 8, wherein the one or more des-31,32-proinsulin ions comprise a des-31,32-proinsulin precursor ion with m/z of 1299.50±0.5.

Para. 10. The method of Para. 8, wherein the one or more des-31,32-proinsulin ions comprise one or more des-31,32-proinsulin fragment ions selected from a group consisting of ions with m/z of 129.20±0.5, 226.20±0.5, and 183.25±0.5.

Para. 11. The method of Para. 6, wherein the metabolic intermediate of proinsulin comprises des-64,65-proinsulin.

Para. 12. The method of Para. 11, wherein the one or more ions comprise one or more des-64,65-proinsulin ions.

Para. 13. The method of Para. 12, wherein the one or more des-64,65-proinsulin ions comprise a des-64,65-proinsulin precursor ion with m/z of 1304.30±0.5.

Para. 14. The method of Para. 12, wherein the one or more des-64,65-proinsulin ions comprise one or more des-64,65-proinsulin fragment ions selected from a group consisting of ions with m/z of 147.15±0.5, 130.15±0.5, and 183.20±0.5.

Para. 15. The method of any one of Paras. 1-14, wherein the analyte comprises a mutant C-peptide.

Para. 16. The method of Para. 15, wherein the mutant C-peptide comprises C-peptide with one additional arginine residue on C-terminal end (C-peptide+R).

Para. 17. The method of Para. 16, wherein the one or more ions comprise one or more C-peptide+R ions.

Para. 18. The method of Para. 17, wherein the one or more C-peptide+R ions comprise a precursor C-peptide+R ion with m/z of 1059.20±0.5.

Para. 19. The method of Para. 17, wherein the one or more C-peptide+R ions comprise one or more C-peptide+R fragment ions selected from a group consisting of ions with m/z of 966.40±0.5, 260.00±0.5, and 844.30±0.5.

Para. 20. The method of Para. 15, wherein the mutant C-peptide comprises C-peptide with two additional arginine residues on C-terminal end (C-peptide+RR).

Para. 21. The method of Para. 20, wherein the one or more ions comprise one or more C-peptide+RR ions.

Para. 22. The method of Para. 21, wherein the one or more C-peptide+RR ions comprise a C-peptide+RR precursor ion with m/z of 1111.50±0.5.

Para. 23. The method of Para. 21, wherein the one or more C-peptide+RR ions comprise one or more C-peptide+RR fragment ions selected from a group consisting of ions with m/z of 896.50±0.5, 499.90±0.5, and 260.30±0.5.

Para. 24. The method of any one of Paras. 1-21, wherein the analyte comprises insulin.

Para. 25. The method of Para. 24, wherein the one or more ions comprise one or more insulin ions.

Para. 26. The method of Para. 25, wherein the one or more insulin ions comprise an insulin precursor ion with m/z of 1162.30±0.5.

Para. 27. The method of Para. 25, wherein the one or more insulin ions comprise one or more insulin fragment ions selected from a group consisting of ions with m/z of 226.20±0.5, 136.10±0.5, and 345.20±0.5.

Para. 28. The method of any one of Para. 1-27, wherein the analyte comprises C-peptide.

Para. 29. The method of Para. 28, wherein the one or more ions comprise one or more C-peptide ions.

Para. 30. The method of Para. 29, wherein the one or more C-peptide ions comprise a C-peptide precursor ion with m/z of 1007.70±0.5.

Para. 31. The method of Para. 29, wherein the one or more C-peptide ions comprise one or more C-peptide fragment ions selected from a group consisting of ions with m/z of 927.50±0.5, 646.40±0.5, and 533.30±0.5.

Para. 32. The method of any one of Paras. 1-31, wherein the analyte is not insulin, C-peptide, or a mixture of insulin and C-peptide.

Para. 33. A method for determining an amount of proinsulin in a sample, the method comprising: immunocapturing proinsulin from the sample; subjecting the immunocaptured proinsulin to an ionization source under conditions suitable to generate one or more proinsulin ions detectable by mass spectrometry; determining an amount of the one or more proinsulin ions by mass spectrometry; and determining the amount of proinsulin in the sample from the amount of the one or more proinsulin ions.

Para. 34. The method of Para. 33, wherein the one or more proinsulin ions comprise a proinsulin precursor ion with a mass-to-charge ratio (m/z) of 1342.20±0.5.

Para. 35. The method of Para. 33 or 34, wherein the one or more proinsulin ions comprise one or more proinsulin fragment ions selected from a group consisting of ions with m/z of 183.30±0.5, 120.20±0.5, and 219.30=0.5.

Para. 36. A method for determining an amount of a metabolic intermediate of proinsulin in a sample, the method comprising: immunocapturing the metabolic intermediate from the sample; subjecting the immunocaptured metabolic intermediate to an ionization source under conditions suitable to generate one or more metabolic intermediate ions detectable by mass spectrometry; determining an amount of the one or more metabolic intermediate ions by mass spectrometry; and determining the amount of the metabolic intermediate of proinsulin in the sample from the amount of the one or more metabolic intermediate ions; wherein the metabolic intermediate of proinsulin is des-31,32-proinsulin, des-64,65-proinsulin, or a mixture thereof.

Para. 37. The method of Para. 36, wherein the metabolic intermediate of proinsulin comprises des-31,32-proinsulin.

Para. 38. The method of Para. 36 or 37, wherein the one or more metabolic intermediate ions comprise one or more des-31,32-proinsulin ions.

Para. 39. The method of Para. 38, wherein the one or more des-31,32-proinsulin ions comprise a des-31,32-proinsulin precursor ion with m/z of 1299.50±0.5.

Para. 40. The method of Para. 38, wherein the one or more des-31,32-proinsulin ions comprise one or more des-31,32-proinsulin fragment ions selected from a group consisting of ions with m/z of 129.20±0.5, 226.20±0.5, and 183.25±0.5.

Para. 41. The method of any one of Paras. 36-40, wherein the metabolic intermediate of proinsulin comprises des-64,65-proinsulin.

Para. 42. The method of any one of Paras. 36-41, wherein the one or more metabolic intermediate ions comprise one or more des-64,65-proinsulin ions.

Para. 43. The method of Para. 42, wherein the one or more des-64,65-proinsulin ions comprise a des-64,65-proinsulin precursor ion with m/z of 1304.30±0.5.

Para. 44. The method of Para. 42, wherein the one or more des-64,65-proinsulin ions comprise one or more des-64,65-proinsulin fragment ions selected from a group consisting of ions with m/z of 147.15±0.5, 130.15±0.5, and 183.20±0.5.

Para. 45. A method for determining an amount of a mutant C-peptide in a sample, the method comprising: immunocapturing the mutant C-peptide from the sample; subjecting the immunocaptured mutant C-peptide to an ionization source under conditions suitable to generate one or more mutant C-peptide ions detectable by mass spectrometry; determining an amount of the one or more mutant C-peptide ions by mass spectrometry; and determining the amount of the mutant C-peptide in the sample from the amount of the one or more mutant C-peptide ions; wherein the mutant C-peptide is C-peptide with one additional arginine residue on C-terminal end (C-peptide+R), C-peptide with two additional arginine residues on C-terminal end (C-peptide+RR), or a mixture thereof.

Para. 46. The method of Para. 45, wherein the mutant C-peptide comprises C-peptide with one additional arginine residue on C-terminal end (C-peptide+R).

Para. 47. The method of Para. 45 or 46, wherein the one or more mutant C-peptide ions comprise one or more C-peptide+R ions.

Para. 48. The method of Para. 47, wherein the one or more C-peptide+R ions comprise a precursor C-peptide+R ion with m/z of 1059.20±0.5.

Para. 49. The method of Para. 47, wherein the one or more C-peptide+R ions comprise one or more C-peptide+R fragment ions selected from a group consisting of ions with m/z of 966.40±0.5, 260.00±0.5, and 844.30±0.5.

Para. 50. The method of any one of Paras. 45-49, wherein the mutant C-peptide comprises C-peptide with two additional arginine residues on C-terminal end (C-peptide+RR).

Para. 51. The method of any one of Para. 45-50, wherein the one or more mutant C-peptide ions comprise one or more C-peptide+RR ions.

Para. 52. The method of Para. 51, wherein the one or more C-peptide+RR ions comprise a C-peptide+RR precursor ion with m/z of 1111.50±0.5.

Para. 53. The method of Para. 51, wherein the one or more C-peptide+RR ions comprise one or more C-peptide+RR fragment ions selected from a group consisting of ions with m/z of 896.50±0.5, 499.90±0.5, and 260.30±0.5.

Para. 54. The method of any one of the preceding Paras., wherein said sample comprises a plasma or serum sample.

Para. 55. The method of any one of the preceding Paras., wherein said ionization source is an electrospray (ESI) ionization source.

Para. 56. The method of any one of the preceding Paras., wherein ionization is in positive ion mode.

Para. 57. The method of any one of the preceding Paras., wherein said sample is subjected to acidic conditions prior to mass spectrometry.

Para. 58. The method of Para. 57, wherein subjecting said sample to acidic conditions comprises subjecting said sample to formic acid.

Para. 59. The method of any one of the preceding Paras., wherein said sample is subjected to basic conditions prior to mass spectrometry.

Para. 60. The method of Para. 59, wherein subjecting said sample to basic conditions comprises subjecting said sample to trizma and/or ethanol.

Para. 61. The method of any one of the preceding Paras., wherein the sample is delipidated prior to quantitation by mass spectrometry.

Para. 62. The method of any one of the preceding Paras., further comprising purifying the sample prior to mass spectrometry.

Para. 63. The method of Para. 62, wherein said purifying comprises subjecting the sample to liquid chromatography.

Para. 64. The method of Para. 63, wherein liquid chromatography comprises high performance liquid chromatography (HPLC) or high turbulence liquid chromatograph (HTLC).

Para. 65. The method of Para. 62, wherein said purifying comprises subjecting a sample to solid phase extraction (SPE).

Para. 66. The method of any one of the preceding Paras., wherein the mass spectrometry is tandem mass spectrometry, high resolution mass spectrometry, or high resolution/high accuracy mass spectrometry.

Para. 67. The method of any one of the preceding Paras., wherein the immunocapture comprises using antibodies.

Para. 68. The method of Para. 67, wherein the antibodies are monoclonal antibodies.

Para. 69. The method of Para. 67 or 68, wherein the antibodies are immobilized on magnetic beads.

Para. 70. The method of any one of the preceding Paras., further comprising washing and eluting the materials immunocaptured on magnetic beads.

While certain embodiments have been illustrated and described, it should be understood that changes and modifications can be made therein in accordance with ordinary skill in the art without departing from the technology in its broader aspects as defined in the following claims.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure

Other embodiments are set forth in the following claims.

What is claimed is:

1. A method for determining an amount of an analyte in a sample by mass spectrometry, the method comprising:
immunocapturing the analyte from the sample;
subjecting the immunocaptured analyte to an ionization source under conditions suitable to generate one or more ions detectable by mass spectrometry;
determining an amount of the one or more ions by mass spectrometry; and
determining the amount of the analyte in the sample from the amount of the one or more ions;
wherein
the analyte comprises insulin, proinsulin, a metabolic intermediate of proinsulin, C-peptide, a mutant C-peptide, or a mixture of any two or more thereof;
the metabolic intermediate of proinsulin is des-31,32-proinsulin, des-64,65-proinsulin, or a mixture thereof; and
the mutant C-peptide is C-peptide with one additional arginine residue on C-terminal end (C-peptide+R), C-peptide with two additional arginine residues on C-terminal end (C-peptide+RR), or a mixture thereof.

2. The method of claim 1, wherein the analyte comprises proinsulin.

3. The method of claim 2, wherein the one or more ions comprise one or more proinsulin ions.

4. The method of claim 3, wherein the one or more proinsulin ions comprise a proinsulin precursor ion with a mass-to-charge ratio (m/z) of 1342.20±0.5.

5. The method of claim 3, wherein the one or more proinsulin ions comprise one or more proinsulin fragment ions selected from a group consisting of ions with m/z of 183.30±0.5, 120.20±0.5, and 219.30±0.5.

6. The method of claim 1, wherein the analyte comprises a metabolic intermediate of proinsulin.

7. The method of claim 6, wherein the metabolic intermediate of proinsulin comprises des-31,32-proinsulin.

8. The method of claim 7, wherein the one or more ions comprise one or more des-31,32-proinsulin ions.

9. The method of claim 8, wherein the one or more des-31,32-proinsulin ions comprise a des-31,32-proinsulin precursor ion with m/z of 1299.50±0.5.

10. The method of claim 8, wherein the one or more des-31,32-proinsulin ions comprise one or more des-31,32-proinsulin fragment ions selected from a group consisting of ions with m/z of 129.20±0.5, 226.20±0.5, and 183.25±0.5.

11. The method of claim 1, wherein the one or more ions comprise one or more des-64,65-proinsulin ions.

12. The method of claim 11, wherein the one or more des-64,65-proinsulin ions comprise a des-64,65-proinsulin precursor ion with m/z of 1304.30±0.5, or wherein the one or more des-64,65-proinsulin ions comprise one or more des-64,65-proinsulin fragment ions selected from a group consisting of ions with m/z of 147.15±0.5, 130.15±0.5, and 183.20±0.5.

13. The method of claim 1, wherein the analyte comprises a mutant C-peptide.

14. The method of claim 13, wherein the mutant C-peptide comprises C-peptide with one additional arginine residue on C-terminal end (C-peptide+R).

15. The method of claim 14, wherein the one or more ions comprise one or more C-peptide+R ions.

16. The method of claim 15, wherein the one or more C-peptide+R ions comprise a precursor C-peptide+R ion with m/z of 1059.20±0.5.

17. The method of claim 15, wherein the one or more C-peptide+R ions comprise one or more C-peptide+R fragment ions selected from a group consisting of ions with m/z of 966.40±0.5, 260.00±0.5, and 844.30±0.5.

18. A method for determining an amount of proinsulin in a sample, the method comprising:
immunocapturing proinsulin from the sample;
subjecting the immunocaptured proinsulin to an ionization source under conditions suitable to generate one or more proinsulin ions detectable by mass spectrometry;
determining an amount of the one or more proinsulin ions by mass spectrometry; and
determining the amount of proinsulin in the sample from the amount of the one or more proinsulin ions.

19. The method of claim 18, wherein the one or more proinsulin ions comprise a proinsulin precursor ion with a mass-to-charge ratio (m/z) of 1342.20±0.5, or wherein the one or more proinsulin ions comprise one or more proinsulin fragment ions selected from a group consisting of ions with m/z of 183.30±0.5, 120.20±0.5, and 219.30±0.5.

20. A method for determining an amount of a mutant C-peptide in a sample, the method comprising:
immunocapturing the mutant C-peptide from the sample;
subjecting the immunocaptured mutant C-peptide to an ionization source under conditions suitable to generate one or more mutant C-peptide ions detectable by mass spectrometry;
determining an amount of the one or more mutant C-peptide ions by mass spectrometry; and
determining the amount of the mutant C-peptide in the sample from the amount of the one or more mutant C-peptide ions;
wherein
the mutant C-peptide is C-peptide with one additional arginine residue on C-terminal end (C-peptide+R), C-peptide with two additional arginine residues on C-terminal end (C-peptide+RR), or a mixture thereof.
